SEQUENCE LISTING (29 sequences)

SEQ ID NO: 1 - RNA, 20 nt, synthetic construct
  misc_feature 1..20: note = Guide RNA
  source 1..20: mol_type = other RNA
aagcgagcaagagaatcccc

SEQ ID NO: 2 - RNA, 76 nt, synthetic construct
  misc_feature 1..76: note = Guide RNA
  source 1..76: mol_type = other RNA
gttttagagctagaaatagcaagttaaaataaggctagtccgttatcaacttgaaaaagtggcaccgagtcggtgc

SEQ ID NO: 3 - protein, 11 aa, synthetic construct
  REGION 1..11: note = Nuclear Localization Signals
GPKKKRKVAAA

SEQ ID NO: 4 - DNA, 20 bp, synthetic construct
  misc_feature 1..20: note = gRNA-InsC-2
  source 1..20: mol_type = other DNA
agcaagagaatccccaggac

SEQ ID NO: 5 - DNA, 20 bp, synthetic construct
  misc_feature 1..20: note = gRNA-InsC-3
  source 1..20: mol_type = other DNA
ttctgtcctggggattctct

SEQ ID NO: 6 - DNA, 76 bp, synthetic construct
  misc_feature 1..76: note = gRNAs scaffold
  source 1..76: mol_type = other DNA
gttttagagctagaaatagcaagttaaaataaggctagtccgttatcaacttgaaaaagtggcaccgagtcggtgc

SEQ ID NO: 7 - RNA, 76 nt, synthetic construct; identical to SEQ ID NO: 2
  misc_feature 1..76: note = gRNAs scaffold
  source 1..76: mol_type = other RNA

SEQ ID NO: 8 - DNA, 24 bp, synthetic construct
  misc_feature 1..24: note = BRCA1 238F Primer
  source 1..24: mol_type = other DNA
acctcctgcattcaaaagattctc

SEQ ID NO: 9 - DNA, 25 bp, synthetic construct
  misc_feature 1..25: note = BRCA1 983R Primer
  source 1..25: mol_type = other DNA
agcaagagagagagagagaaagaca

SEQ ID NO: 10 - DNA, 20 bp, synthetic construct
  misc_feature 1..20: note = BRCA1
  source 1..20: mol_type = other DNA
aaagcgagcaagagaatccc

SEQ ID NO: 11 - DNA, 20 bp, synthetic construct
  misc_feature 1..20: note = Ex19-5382-InsC
  source 1..20: mol_type = other DNA
aagcgagcaagagaatcccc

SEQ ID NO: 12 - DNA, 180 bp, synthetic construct
  misc_feature 1..180: note = ssODN
  source 1..180: mol_type = other DNA
tttggtttctttcagcatgattttgaagtcagaggagatgtggtcaatggaagaaaccaccaaggtccaaagcgagcaagagaatccccaggacagaaaggtaaagctccctccctcaagttgacaaaaatctcaccccaccactctgtattccactcccctttgcagagatgggccgct

SEQ ID NO: 13 - DNA, 85 bp, synthetic construct
  misc_feature 1..85: note = Ex19-5382-insC
  source 1..85: mol_type = other DNA
catgattttgaagtcagaggagatgtggtcaatggaagaaaccaccaaggtccaaagcgagcaagagaatccccaggacagaaag

SEQ ID NO: 14 - DNA, 1766 bp, synthetic construct
  misc_feature 1..1766: note = Ex19-5382-InsC Reporter System
  source 1..1766: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagccaccaatgcatgattttgaagtcagaggagatgtggtcaatggaagaaaccaccaaggtccaaagcgagcaagagaatccccaggacagaaagaccggtcgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaagcggccgca

SEQ ID NO: 15 - RNA, 23 nt, synthetic construct
  source 1..23: mol_type = other RNA
aagcgagcaagagaatccccaag

SEQ ID NO: 16 - DNA, 24 bp, synthetic construct
  source 1..24: mol_type = other DNA
aaagcgagcaagagaatccccagg

SEQ ID NO: 17 - DNA, 26 bp, synthetic construct
  source 1..26: mol_type = other DNA
aaagcgagcaagagaatccccaggac

SEQ ID NO: 18 - DNA, 26 bp, synthetic construct
  source 1..26: mol_type = other DNA
aaagcgagcaagagaatcnncccagg

SEQ ID NO: 19 - DNA, 26 bp, synthetic construct
  source 1..26: mol_type = other DNA
aaagcgagcaagagaatcncccagga

SEQ ID NO: 20 - DNA, 24 bp, synthetic construct
  source 1..24: mol_type = other DNA
aaagcgagcaagagaatccaggac

SEQ ID NO: 21 - DNA, 23 bp, synthetic construct
  source 1..23: mol_type = other DNA
aaagcgagcaagagaatcccagg

SEQ ID NO: 22 - DNA, 28 bp, synthetic construct
  source 1..28: mol_type = other DNA
ccaaagcgagcaagagaatccccaggac

SEQ ID NO: 23 - DNA, 27 bp, synthetic construct
  source 1..27: mol_type = other DNA
ccaaagcgagcaagagaatcccaggac

SEQ ID NO: 24 - DNA, 23 bp, synthetic construct
  source 1..23: mol_type = other DNA
agcaagagaatccccaggacaga

SEQ ID NO: 25 - DNA, 23 bp, synthetic construct
  source 1..23: mol_type = other DNA
ttctgtcctggggattctcttgc

SEQ ID NO: 26 - DNA, 24 bp, synthetic construct
  source 1..24: mol_type = other DNA
aaagcgagcaagagaatccccggg

SEQ ID NO: 27 - DNA, 23 bp, synthetic construct
  source 1..23: mol_type = other DNA
aaagcgagcaagaaatccccagg

SEQ ID NO: 28 - DNA, 24 bp, synthetic construct
  source 1..24: mol_type = other DNA
aaagcgagcaagagaatcccaagg

SEQ ID NO: 29 - RNA, 24 nt, synthetic construct
  source 1..24: mol_type = other RNA
aaagcgagcaagagaatccccggg

(19) United States
(12) Patent Application Publication
(10) Pub. No.: US 2023/0069642 A1
(43) Pub. Date: Mar. 2, 2023

(54) PRECISE TEMPLATE-FREE CORRECTION OF BRCA1 MUTATION IN HUMAN CELLS VIA GENOME EDITING

(71) Applicant: New York University, New York, NY (US)

(72) Inventors: Chengzu Long, New York, NY (US); Orrin Devinsky, New York, NY (US); Gabriella Rudy, New York, NY (US)

(21) Appl. No.: 17/819,547

(22) Filed: Aug. 12, 2022

Related U.S. Application Data
(60) Provisional application No. 63/232,598, filed on Aug. 12, 2021.

(51) Int. Cl. C07K 14/47; C12N 15/11; C12N 5/074; C12N 5/09

(52) U.S. Cl. CPC C07K 14/4748; C12N 15/111; C12N 5/0607; C12N 5/0693; C12N 2310/20

(57) ABSTRACT

Provided are compositions and methods used for precise template-free correction of BRCA1 mutation in human cells via genome editing. The method involves modifying DNA that includes a BRCA1-5382-InsC mutation by introducing into cells comprising the BRCA1-5382-InsC a Cas enzyme and a guide RNA. A guide RNA that produces improved results relative to other guide RNAs is provided. Also provided are modified stem cells that contain an introduced BRCA1-5382-InsC mutation.

### CROSS REFERENCE TO RELATED APPLICATIONS

This application claims priority to U.S. provisional patent application No. 63/232,598, filed Aug. 12, 2021, the entire disclosure of which is incorporated herein by reference.

### SEQUENCE LISTING

The instant application contains a Sequence Listing which has been submitted in .xml format and is hereby incorporated by reference in its entirety. Said .xml copy was created on Aug. 12, 2022, is named “05863600523.xml.” and is 33,930 bytes in size.

### BACKGROUND

Cancer is the second leading cause of death globally, and prevention offers the greatest opportunity to reduce morbidity and mortality. Gene therapies for cancer have typically focused on therapy after the cancer has developed, such as chimeric antigen receptor T cells (CAR-T cells). By contrast, genetic therapies that “correct” oncogenic variants have not been actively pursued. The Clustered Regularly Interspaced Short Palindromic Repeat/Cas9 (CRISPR/Cas9) system is an invaluable research tool that is currently undergoing therapeutic trials related to several diseases (e.g., sickle cell, liver and eye diseases) due to its efficiency in targeted and site-specific correction of genetic mutations. If CRISPR/Cas9-mediated gene editing can successfully correct oncogenic mutations, it could potentially prevent the initiation, growth and spread of cancer. Advancements in reprogramming technologies have led to the development of human induced pluripotent stem cells (iPSCs) as a new tool to model disease and discover drugs. iPSCs can undergo efficient gene editing and reprogramming, making them excellent models of genetic diseases. This strategy has revolutionized our understanding of genetic risk factors that predispose individuals to certain cancers and could help develop novel therapeutic strategies to permanently prevent oncogenesis.

Breast cancer is one of the most diagnosed cancers among US women and is the second leading cause of death among women, globally. Genetic mutations in the two major breast cancer susceptibility genes, BRCA1 and BRCA2, are the major cause of hereditary breast cancer and account for ˜3% of all breast cancers (2). BRCA gene mutations are also associated with an increased risk of breast and prostate cancer in men, and pancreatic cancer and melanoma in both sexes. BRCA1 and BRCA2 are tumor-suppressor genes (TSGs) vital for homology directed repair (HDR) of DNA double-stranded breaks (DSBs). They are crucial for maintaining genomic stability. Women carrying pathogenic germline mutations in either BRCA1 or BRCA2 are much more susceptible to developing breast and ovarian cancers by age 70; their risk is increased by 10-20-fold. Ashkenazi Jews commonly harbor BRCA1 or BRCA2 mutations, with approximately 1:40 individuals harboring one of three oncogenic founder mutations: BRCA1-185-delAG, BRCA1-5382-InsC, and BRCA2-6174-delT. Many women with these mutations elect to undergo bilateral prophylactic (preventative) mastectomy, as well as removal of their ovaries. However, there is an unmet need for safer and less invasive prevention strategies. The present disclosure is pertinent to this need.

### BRIEF SUMMARY

The present disclosure provides compositions and methods that are used for precise template-free correction of BRCA1 mutation in human cells via genome editing. In an embodiment, the disclosure provides a method for modifying DNA comprising a BRCA1-5382-InsC mutation. This approach comprises introducing into cells comprising the BRCA1-5382-InsC a Cas enzyme and a guide RNA. The method is performed in a DNA repair template free manner. Thus, the method can be performed without introducing a single stranded DNA or a double stranded DNA that is introduced into a chromosome to correct the BRCA1-5382-InsC mutation. In embodiments, the guide RNA is functional with an AGG protospacer adjacent motif (PAM) that is proximal to the BRCA1-5382-InsC mutation. By “functional” it is meant that in the presence of the particular guide RNA and a Cas enzyme the BRCA1-5382-InsC mutation is eliminated. In embodiments, the functional guide RNA comprises the sequence AAGCGAGCAAGAGAAUCCCC (SEQ ID NO: 1).

The method can be performed in any cells, in vitro or in vivo, that harbor the BRCA1-5382-InsC mutation. In non-limiting embodiments, such cells are breast cancer cells, ovarian cancer cells, prostate cancer cells, or melanoma cells. In embodiments, the method is performed by introducing a described system into cells of an individual who is suspected of having, has been diagnosed with, or is at risk for developing, any of the aforementioned cancer types. In embodiments, use of the compositions and methods inhibitors recurrence or metastasis of cancer.

In embodiments, some or all of the components of the described system can be introduced into an individual by using a single, or more than one, expression vector that encodes and expresses some or all of the components. In embodiments, a complex comprising the Cas enzyme and the guide RNA is introduced into cells.

In embodiments, the BRCA1-5382-InsC mutation is eliminated by Canonical Non-homologous end joining (c-NHEJ). In embodiments, elimination of the BRCA1-5382-InsC mutation reverses a loss of heterozygosity for the BRCA1 allele.

The disclosure also provides modified cells, such as any type of stem cells, into which a BRCA1-5382-InsC mutation has been introduced. In embodiments, the stem cells induced pluripotent stem cells (iPSCs). In embodiments, the introduced the BRCA1-5382-InsC mutation is a heterozygous mutation. Such modified cells may be present in an in vitro in vitro cell culture, and may be used to produce non-human animal models for use in evaluating properties of cells that contain the the BRCA1-5382-InsC, and for evaluating therapeutic approaches that target the mutation or cells that contain the mutation.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Sequences in Figures: The portion of the sequence in bold and capital on FIG. 1 panel A (Ex19-5382-InsC), FIG. 2 panel A (WT), and FIG. 3 panel A (Ex19-5382-InsC), is SEQ ID NO: 13. The sequence in FIG. 2 panel A (InsC) is SEQ ID NO: 12. The sequence in FIG. 1 panel A (gRNA-InsC) is SEQ ID NO: 15. The sequence in FIG. 1 panel C (eGFD+/tdTom+) and (eGFD+/tdTom−) control, and FIG. 4 panel B ((gRNA-InsC-3-mix), and FIG. 4 panel B ((gRNA-InsC-2-mix(0)), and FIG. 4 panel B ((gRNA-InsC-3-mix(0)), and FIG. 1 panel C (3n+1) (0-78%), is SEQ ID NO: 16. The sequence in FIG. 1 panel C (3n+1) (0-78%) is SEQ ID NO: 17. The sequence in FIG. 1 panel C (3n) is SEQ ID NO: 18. The sequence in FIG. 1 panel C (3n+2) is SEQ ID NO: 19. The sequence in FIG. 1 panel C (3n+2)(with hyphen) is SEQ ID NO: 20. The sequence in FIG. 2 panel B (gRNA-WT), and FIG. 3 panel A (gRNA-InsC), is SEQ ID NO: 21. The sequence in FIG. 2 panel B (ss-ODN), and FIG. 2 panel C (Heterozygous Knock-in), and in FIG. 2 panel D (Knock-in:4/7), is SEQ ID NO: 22. The sequence in FIG. 2 panel C (WT), and FIG. 2 panel D (WT:3/7), is SEQ ID NO: 23. The sequence in FIG. 4 panel A (gRNA-InsC-2) is SEQ ID NO: 24. The sequence in FIG. 4 panel A (gRNA-InsC-3) is SEQ ID NO: 25. The sequence in FIG. 4 panel B ((gRNA-InsC-2-mix), and FIG. 4 panel B ((gRNA-InsC-3-mix), is SEQ ID NO: 26. The sequence in FIG. 1 panel C (3n+1) (0-100%), and FIG. 3 panel C (Knock-in InsC mix (control)), and FIG. 3 panel C (InsC), and FIG. 3 panel C (Corr. Mix.) and InsC, and FIG. 3 panel C (Corr. Mix.)wt, and FIG. 4 panel B ((gRNA-InsC-3-mix(−1)), and FIG. 4 panel B (control mix (0)), is SEQ ID NO: 27. The sequence in FIG. 3 panel C (WT), and FIG. 3 panel C (Corr. Mix.), and FIG. 3 panel C (Corr.-sc4), and FIG. 3 panel C (Corr.-sc4)wt, is SEQ ID NO: 28. The sequence in FIG. 4 panel B ((gRNA-InsC-2-mix(−1)), and FIG. 4 panel B (control mix (−1)), and FIG. 4 panel B (control mix), is SEQ ID NO: 29.

FIG. 1. Panel A. Schematic of CRISPR/Cas9-mediated correction of BRCA1-5382-InsC in a reporter system. Reporter plasmid-InsC (upper plasmid) was generated by cloning BRCA1 Ex19-5382-InsC into a plasmid containing a CMV promoter and tdTomato (tdTom) fluorescent protein, out-of-frame with respect to tdTom. This causes a premature stop codon (represented by the stop sign) and a lack of tdTom expression (represented by a grey box). Following CRISPR/Cas9-mediated correction of Ex19-5382-insC, the open reading frame was restored and tdTom expression was rescued (represented by a glowing red box in the lower plasmid). Corrected BRCA1 exon 19 (termed “Ex19”) is represented by a box in the lower plasmid (labeled “Reporter plasmid-corr.”). Ex19-5382-InsC (upper sequence) is shown, with the InsC mutation underlined. The 20 base pair gRNA target sequence of BRCA1-5382-InsC (labeled “gRNA-InsC”) is shown (lower sequence). The underlined text marks the mutated site, and the black arrowhead marks the Cas9 cleavage site. The NGG PAM is labeled with underlined text.

FIG. 1. Panel B. Strategy for BRCA1-5382-InsC correction using a reporter system in HEK293T cells. The reporter plasmid (upper plasmid) contains Ex19-5382-InsC and tdTom. The rescue plasmid contains Cas9, eGFP, and gRNA-InsC. The rescue plasmid and reporter plasmid (together termed “reporter system”) were co-transfected into HEK293T cells. Transfected cells were sorted based on eGFP and tdTom fluorescence.

FIG. 1. Panel C. Sanger sequencing results (upper box for each sample) and Synthego Inference of CRISPR Edits (ICE)) (ice.synthego.com/) analysis (lower box for each sample) corresponding to PCR-amplified DNA that was extracted from transfected cells. The effect of each Cas9-induced indel, with respect to the open reading frame shift, is shown to the left of the sequencing data. “3n” indicates an in-frame sequence. “3n+1” and “3n+2” indicate a sequence out-of-frame by 1 and 2 base pairs respectively. The upper ICE analysis and sequencing results correspond to the eGFP+/tdTom+ cell mixture, within which ˜78% of alleles have a 1 base pair frame shift, 15% have a restored reading frame, and 5% have a 2 base pair frame shift. The lower ICE analysis and sequencing results correspond to the eGFP+/tdTom− (control) cell mixture, within which 100% of alleles contain a 1 base pair frame shift. In both ICE analyses, the dashed line represents the Cas9 cleavage site and an “N” or hyphen (adjacent to the dashed line) indicates an insertion and a deletion, respectively.

FIG. 1. Panel D. Efficiency of CRISPR/Cas9-mediated correction of BRCA1-5382-InsC in 3 independent reporter system transfections (n=3). The ratio of InsC mutations:indels in the target region is shown for eGFP+/tdTom+ (left column) and eGFP+/tdTom− (right column) cell mixtures, among all 3 trials.

FIG. 2. Panel A. Schematic of BRCA1 exon 19, showing the WT (upper) and mutant (lower) sequences within the target region. The InsC mutation in exon 19 and the premature stop codon in exon 21 are underlined.

FIG. 2. Panel B. Schematic of the 20 base pair gRNA target sequence of BRCA1 (labeled “gRNA-WT”) is shown (upper sequence). The black arrowhead marks the Cas9 cleavage site and the NGG PAM is labeled with underlined text. Schematic of the 180 base pair ssODN is shown (lower sequence), containing the InsC mutation (underlined). The sequence on with FIG. 2, panel A (InsC), are SEQ ID NO: 12 and SEQ ID NO: 13.

FIG. 2. Panel C. Sanger sequencing results corresponding to a WT (upper) and a heterozygous knock-in (lower) single clone, following transfection with a plasmid containing only eGFP and knock-in plasmid, respectively. Corresponding amino acid residues are shown above each sequence. Mutated residues are marked in bold text.

FIG. 2. Panel D. TOPO-TA cloning and Sanger sequencing results corresponding to individual alleles of heterozygous knock-in single clone #4 (termed “Heterozygous Knock-in InsC-SC4”). The raw count of each allele is shown to the left of the sequencing data. Corresponding amino acid residues are located above each sequence. The mutated residues are marked in bold text. The red arrowhead indicates the InsC mutation.

FIG. 3. Panel A. Schematic of the targeted exon 19 with BRCA1-5382-InsC (the InsC mutation is indicated by a red arrowhead) and mutant sequence (upper sequence). The InsC mutation in exon 19 is underlined. The premature stop codon in exon 21 is represented by the ‘STOP,’ which is shown in underlined text. The 20 base pair gRNA target sequence of BRCA1-5382-InsC (labeled “gRNA-InsC”) is shown (lower sequence). The InsC mutation is underlined. The PAM is marked by the ‘PAM,’ which is underlined, and the Cas9 cleavage site is indicated by a black arrowhead.

FIG. 3. Panel B. Schematic of the template-free correction of BRCA1-5382-InsC via c-NHEJ. The red arrowhead and stop sign in the upper gene model indicate the InsC mutation and the premature stop codon, respectively. Following Cas9-mediated DNA cleavage (represented by scissors), the c-NHEJ pathway is activated, resulting in the correction of BRCA1-5382-InsC (lower left gene model, labeled “Corr.”). The MMEJ pathway generates indels (>2 bp) within the target region, (represented by a black box in exon 19 in the lower middle gene model, termed “Indels”), but is impaired due to insufficient regions of homology flanking the cut site. The HDR pathway is impaired by the partial absence of BRCA1, which plays an essential role in this pathway. In the lower right gene model (labeled “InsC”), the red arrowhead marks the InsC mutation site in exon 19, and the stop sign represents the premature stop codon in exon 21.

FIG. 3. Panel C. Sanger sequencing results and corresponding ICE analysis following transfection of Heterozygous Knock-in InsC-SC4 with a plasmid containing only eGFP (upper sample) and those transfected with the rescue plasmid (middle and lower samples). The ICE analyses show the percentage of WT and mutant alleles, labeled WT and InsC, respectively. The upper 2 samples (labeled “Knock-in InsC-mix (control)” and “Corr.-mix”) correspond to cell mixtures, and the lower sample (labeled “Corr.-SC4”) corresponds to a corrected single clone.

FIG. 3. Panel D. Efficiency of CRISPR/Cas9-mediated, template-free correction of BRCA1-5382-InsC in iPSCs (n=3). The ratio of corrected:uncorrected alleles in the cell mixtures isolated from each trial are shown in the left column (labeled “Corr.-mix-WT:InsC”). The number of single clones that were corrected out of the total number of single clones isolated from each trial is shown in the right column (labeled “Corr.-SC count”).

FIG. 4. Panel A. Pool of gRNA target sequences of BRCA1-5382-InsC tested, but not ultimately used, for the template-free correction of knock-in BRCA1-5382-InsC via c-NHEJ-mediated gene editing (upper sequences, termed “gRNA-InsC-2” and “gRNA-InsC-3”). Schematic of the target region with entire pool of gRNAs tested for the template-free correction of BRCA1-5382-InsC. The mutation site is underlined (lower schematic).

FIG. 4. Panel B. Sanger sequencing results (upper boxes for each sample) and corresponding ICE analyses (lower box for each sample) following transfection of Heterozygous Knock-in InsC-SC4 with a plasmid containing gRNA-InsC-2 (upper sample, labeled “gRNAInsC-2-Mix”), gRNA-InsC-3 (middle sample, labeled “gRNA-InsC-3-Mix”) or only eGFP (lower sample, labeled “Control-Mix”). The ICE analyses for gRNAInsC-2-Mix and gRNA-InsC-3-Mix show the cleavage site (represented by a dashed vertical line) produced by gRNA-InsC-2 and gRNA-InsC-3, respectively. The ICE analysis for Control-Mix shows the cleavage site produced by “gRNA-WT,” indicating that no editing occurred.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

The disclosure includes all polynucleotide and all amino acid sequences that are identified herein by way of a database entry. Such sequences are incorporated herein by reference as they exist in the database on the filing date of this application or patent.

In embodiments, the disclosure provides compositions, methods, one or more guide RNAs, and CRISPR systems for correcting BRCA mutations. In one aspect the disclosure provides for CRISPR-based, template-free correction of the BRCA1-5382-InsC mutation. The chromosomal location of the BRCA1-5382-InsC mutation is known in the art. This mutation contains a single nucleotide insertion in exon 19 (termed “Ex19”) that disrupts the open reading frame, leading to a premature stop codon in exon 21 (termed “Ex21”) and a truncated protein product. One functional allele is generally sufficient to maintain BRCA1's regulatory function. However, loss of heterozygosity (LOH), which TSG's are particularly susceptible to, results in a loss-of-function variant. In this regard, when expressed, truncated BRCA1 ceases to regulate HDR, causing additional mutations to accumulate throughout the genome. This accumulation characterizes genomic instability, a hallmark cancer risk (7). Conventional gene editing systems generally repair mutations following disease onset, however, the present disclosure provides for correcting BRCA1-5382-InsC prior to LOH, which thereby would prevent genomic instability and decrease the likelihood of oncogenesis. Thus, in embodiments, the disclosure provides for preventing LOH, or correction of LOH, i.e., restoring one functional BRCA1 allele.

By designing the described system with an enhanced probability of generating preferred mutations, the disclosure includes generation of isogenic patient cells with greater efficiency as compared to traditional HDR methods. Thus, the present disclosure provides compositions and methods for producing precise correction of the described mutations.

In embodiments, a Cas enzyme is used in combination with a single guide RNA that targets the BRCA1-5382-InsC in an AGG protospacer adjacent motif (PAM) dependent manner. The PAM is generally located within 2-6 nucleotides downstream of the DNA sequence targeted by the guide RNA and the Cas cuts 3-4 nucleotides upstream of the PAM.

In an embodiment, the guide RNA comprises the sequence5′-AAGCGAGCAAGAGAAUCCCC-3′ (SEQ ID NO: 1). The C in bold corresponds to the C that is deleted in the BRCA1-5382-InsC mutation. This guide RNA is referred to herein as gRNA-“InsC” and “InsC-1.” As demonstrated in the Examples below, this gRNA is effective to correct the BRCA1-5382-InsC mutation, whereas use of CRISPR systems with other guide RNAs that target the same mutation are not, or are not as efficient as a CRISPR system that includes the InsC-1 gRNA. Thus, in embodiments, the disclosure provides for increased frequency of correction of BRCA1-5382-InsC, such as within a population of cells, relative to a control. The control can be any suitable control, such as a reference value. In embodiments, the reference value comprises a standardized curve(s), a cutoff or threshold value, and the like. In embodiments, increased correction efficiency comprises use of a system of this disclosure to correct the described BRCA1 mutation in a population of cells using the InsC-1 guide RNA relative to the same approach using a control guide RNA, representative and non-limiting embodiments of which are shown in the Examples and include guide RNAs comprising the sequence shown for gRNA-InsC-2 and gRNA InsC-3.

In embodiments, a guide RNA comprising the InsC-1 sequence (and/or a control guide RNA) is present in a single RNA polynucleotide that further comprises a scaffold sequence, a non-limiting embodiment of which comprises the sequence5′-guuuuagagcuagaaauagcaaguuaaaauaaggcuaguccguuaucaacuugaaaaaguggcaccgagucggugc-3′ (SEQ ID NO: 2).The described sequence may be at the 3′ end of the InsC-1 guide RNA sequence which is5′-AAGCGAGCAAGAGAAUCCCC (SEQ ID NO: 1).

In embodiments, the disclosure comprises introducing into cells comprising the BRCA1-5382-InsC a CRISPR system comprising a Cas nuclease and a guide RNA comprising the InsC-1 sequence to thereby correct the mutation. In embodiments, correction of the mutation comprises a single nucleotide deletion that restores a single nucleotide deletion in exon 19 of the human BRCA1 gene, which in turn eliminates a stop codon in the open reading frame of exon 21 of the human BRCA1 gene. In embodiments, a C is deleted in the mutation that is corrected by the disclosed approaches. In embodiments, correction (e.g., deletion of the inserted C in exon 19) results in production of a non-truncated protein encoded by the human BRCA1 gene.

The method is performed in a DNA template-free manner, meaning no exogenous single-stranded or double stranded DNA template is introduced into the cells for the purpose of insertion of the exogenous DNA into a chromosome. Thus, the method is DNA repair template-free.

In embodiments, the individual in which the BRCA1-5382-InsC mutation present is at risk for developing cancer, is suspected of having, or has been diagnosed with cancer. In embodiments, the cancer is breast cancer, ovarian cancer, prostate cancer, or melanoma. In embodiments, the disclosure comprises identification of an individual as having cells comprising the BRCA1-5382-InsC mutation, and introducing the described CRISPR system into the cells to correct the mutation. In embodiments, the described correction comprises Canonical Non-homologous end joining (c-NHEJ) whereby a single nucleotide is deleted and an open reading frame is restored. In embodiments, the disclosure further comprises confirming the deletion of the insertion by sequencing a segment of a chromosome comprising the deletion site.

In an embodiment, a Cas protein used in this disclosure comprises one or more nuclear localization signals (NLSs). In an embodiment, the one or more NLSs comprise the sequence: GPKKKRKVAAA (SEQ ID NO: 3).

In specific and non-limiting embodiments, the Cas comprises a Cas9, such as Streptococcus pyogenes (SpCas9). Derivatives of Cas9 are known in the art and may also be used with the described DNA polymerase. Such derivatives may be, for example, smaller enzymes that Cas9, and/or have different proto adjacent motif (PAM) requirements. In a non-limiting embodiment, the Cas enzyme may be Cas12a, also known as Cpf1, or SpCas9-HF1, or HypaCas9, or xCas9, or Cas9-NG.

The reference sequence of S. pyogenes is available under GenBank accession no. NC_002737, with the Cas9 gene at position 854757-858863. The S. pyogenes Cas9 amino acid sequence is available under number NP_269215. These sequences are incorporated herein by reference as they were provided in the database on the priority date of this application or patent.

Any of the described components may be introduced into cells using any suitable route and form. In embodiments, the disclosure provides for use of one or more plasmids or other suitable expression vectors that encode the targeting RNA, and/or the described proteins. In embodiments, a single plasmid encodes a Cas nuclease and a guide RNA. In embodiments, one plasmid encodes the Cas nuclease and a separate plasmid encodes the guide RNA. In embodiments, the disclosure provides RNA-protein complexes, e.g., RNAPs, which can be introduced into cells. RNAPs can be assembled if desired in vitro prior to introduction into cells.

In embodiments, a viral expression vector may be used for introducing one or more of the components of the described system. Viral expression vectors may be used as naked polynucleotides, or may comprise viral particles. In embodiments, the expression vector comprises a modified viral polynucleotide, such as from an adenovirus, a herpesvirus, or a retrovirus, such as a lentiviral vector. In embodiments, one or more components of the described systems may be delivered to cells using, for example, a recombinant adeno-associated virus (rAAV) vector. rAAV vectors are commercially available, such as from TAKARA BIO® and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure.

In embodiments, non-viral delivery systems may be used for introducing one or more of the components of the described system. Non-viral tools including hydrodynamic injection, electroporation and microinjection. Hydrodynamic injection can systemically deliver the described systems into targeted tissues, including but not necessarily limited to cancer cells. Chemical vectors, such as lipids and nanoparticles, are widely used for delivery. Cationic lipids interact with negatively charged DNA and the cell membrane, protecting the DNA and cellular endocytosis. DNA nanoparticles, are potential delivery strategies. DNA conjugated to gold nanoparticles (CRISPR-gold) complexed with cationic endosomal disruptive polymers can deliver the described systems into human cells.

In embodiments, a described CRISPR system may be introduced systemically, such as by intravenous administration, or may be introduced directly into a tumor.

In embodiments, expression vectors, proteins, ribonucleoproteins (RNPs), polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients, and stabilizers can be found, for example, in Remington: The Science and Practice of Pharmacy (2005) 21st Edition, Philadelphia, Pa. Lippincott Williams & Wilkins, the disclosure of which is incorporated herein by reference. Further, any of a variety of therapeutic delivery agents can be used, and include but are not limited to nanoparticles, lipid nanoparticle (LNP), fusosomes, exosomes, and the like. In embodiments, a biodegradable material can be used. In embodiments, poly(lactide-co-galactide) (PLGA) is a representative biodegradable material, but it is expected that any biodegradable material, including but not necessarily limited to biodegradable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In embodiments, a combination of proteins, and a combination of one or more proteins and polynucleotides described herein, may be first assembled in vitro and then administered to a cell or an organism.

In an embodiment, the disclosure provides an isogenic model of a BRCA mutation developed in stem cells. In an embodiment, the stem cells comprise induced pluripotent stem cells (iPSCs). In embodiments, the disclosure provides for iPSC isogenic models, in which the wild type and mutant cells are genetically identical, except for the mutation, which allows distinguishing the effects of pathogenic mutations from those due to the cell's genetic background. In embodiments, non-human mammals produced using the stem cells are provided. Thus, in embodiments, the disclosure provides modified stem cells comprising an introduced BRCA1-5382-InsC mutation. “Introduced” as used herein means the stem cells did not have any copies of the BRCA1-5382-InsC mutation before being modified using the described compositions and methods.

The disclosure includes all reporter constructs and cells comprising the reporter constructs, described herein. Substitutions of the type of fluorescent markers used in the reporter constructs will be apparent to those skilled given the benefit of this disclosure, in the art and are encompassed by this disclosure.

The disclosure is illustrated by the following Examples, which are not intended to limit the scope of the disclosure. In particular, the Examples demonstrate precise correction of the BRCA1-5382-InsC mutation in human iPSCs via CRISPR/Cas9-mediated template-free gene editing using a specific guide RNA. This approach can be extended to correction of this mutation in human cells that contain this mutation, as demonstrated in subsequent Examples.

### EXAMPLE 1

This Example provides a non-limiting proof-of-concept demonstration designed to test the efficacy of an aspect of the described strategy in a reporter system. We cloned mutated Ex19 (termed “Ex19-5382-InsC”) into a Lentivirus (pLenti) backbone to generate a recombinant plasmid (termed “reporter plasmid”). pLenti expresses a CMV promoter, tdTomato fluorescent protein (tdTom) and enhanced Green Fluorescent Protein (eGFP). Ex19-5382-InsC was inserted immediately downstream of the CMV promoter and out-of-frame with tdTom, resulting in a premature stop codon in tdTom and preventing its expression. Upon CRISPR/Cas9-mediated correction of the open reading frame, tdTom expression was rescued. This cloning strategy served as an accurate and quantifiable method to visualize correction efficacy using a reporter system.

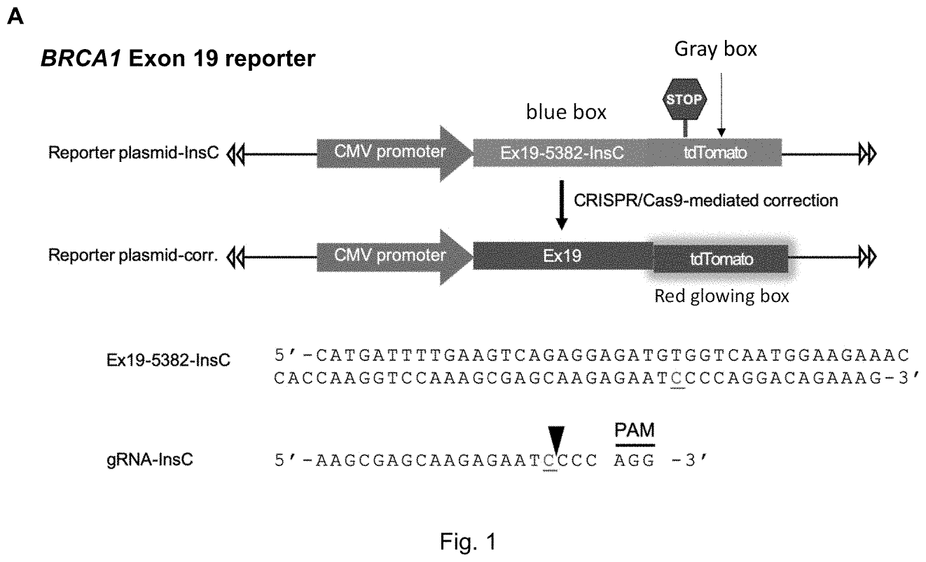

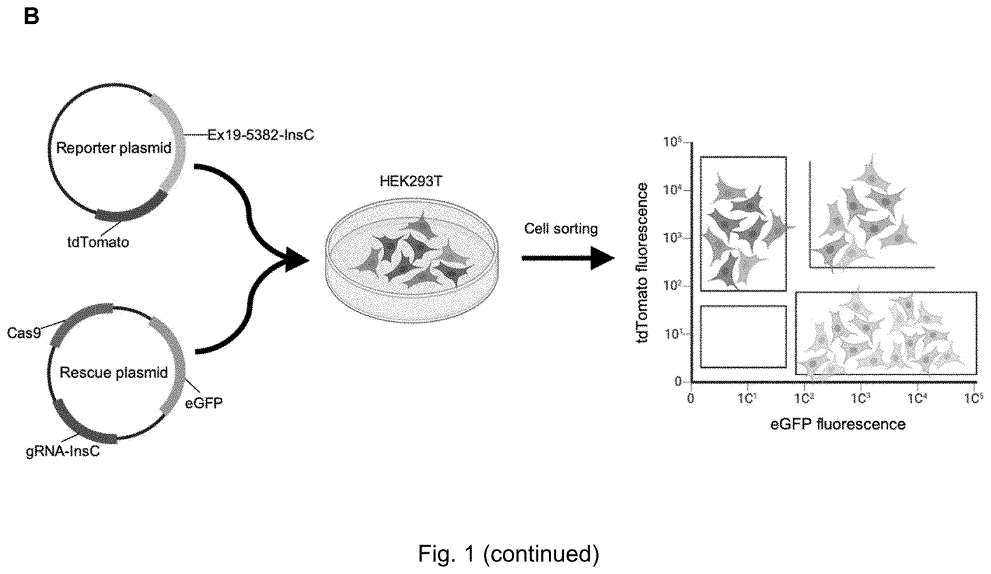

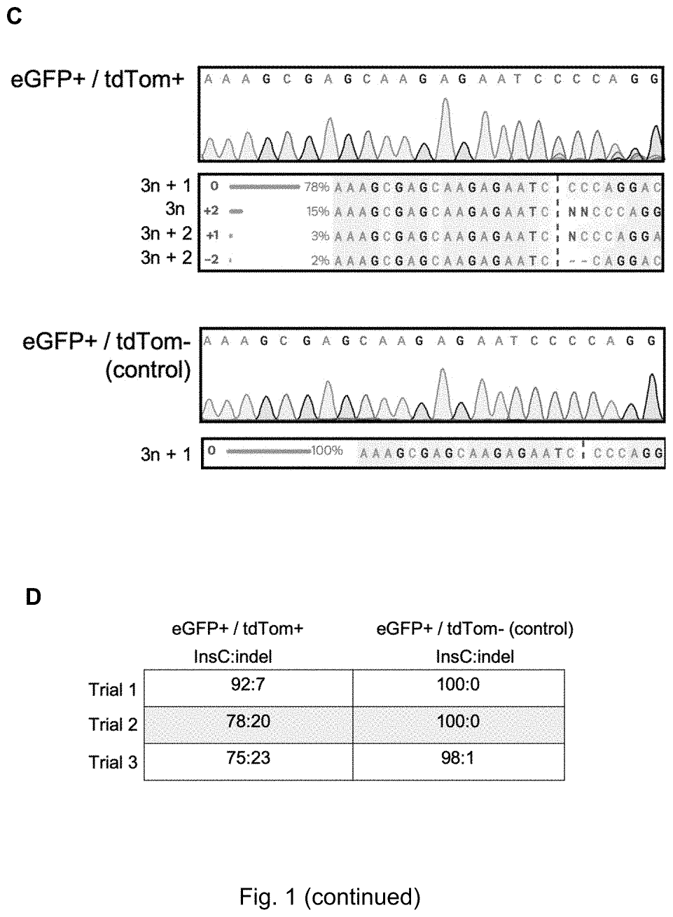

Next, a plasmid (referred to herein as a “rescue plasmid”) was designed to correct the mutation within the reporter plasmid. The rescue plasmid was constructed by cloning a gRNA targeting the region of interest in BRCA1-5382-InsC (termed “gRNA-InsC”), into a Cas9 plasmid expressing enhanced Green Fluorescent Protein (eGFP) (FIG. 1, panel A). The reporter and rescue plasmid (referred to herein as a “reporter system”) were co-transfected into human embryonic kidney 293T (HEK293T) cells. The complementarity between gRNA-InsC and the target sequence facilitates system localization to the mutation site. Subsequently, Cas9 nuclease induces a DNA DSB 3 base pairs upstream of the PAM. Upon detection of the DSB, the cell initiates a repair pathway to fix the damage.

Following reporter system transfection, cells were sorted based on their expression of eGFP (eGFP+) and tdTom (tdTom+) fluorescence (FIG. 1, panel B). Expression of eGFP alone (eGFP+/tdTom−) indicates successful transfection of the rescue plasmid without a reading frame correction. Co-expression of eGFP and tdTom (eGFP+/tdTom+) indicates successful transfection and open reading frame correction. Cells lacking fluorescence (eGFP−/tdTom−) did not undergo successful transfection or reading frame correction. Immediately following sorting, eGFP+/tdTom+ and eGFP+/tdTom− cells were isolated. DNA from the cell mixtures was extracted and amplified via polymerase chain reaction (PCR) and sent for Sanger sequencing. Sequencing results and corresponding Synthego Inference of CRISPR Edits (ICE) (ice.synthego.com/) analysis revealed the efficacy of correcting BRCA1-5382-InsC in the reporter system (FIG. 1, panel C). Within the eGFP+/tdTom+ cell mixture, the reading frame remained unchanged in 78% of alleles, corrected in 15% of alleles, and uncorrected with the addition of indels in 5% of alleles. Within the eGFP+/tdTom− cell mixture, the reading frame remained uncorrected in 100% of alleles, which indicates that a lack of tdTom expression corresponds to an uncorrected reading frame. This demonstrates accuracy of the described approach.

We repeated the experiment in 3 independent trials (n=3) (FIG. 1, panel D). In each trial, the ratio of unedited:edited alleles was compared between the eGFP+/tdTom+ and eGFP+/tdTom− cell mixtures. In all 3 trials, the eGFP+/tdTom+ cell mixture had a higher ratio of unedited:edited alleles, in comparison to the eGFP+/tdTom− cell mixture. These results support correction of BRCA1-5382-InsC using template-free, CRISPR/Cas9-mediated gene editing. Thus, this Example provides a non-limiting proof-of-concept that the described rescue plasmid can correct BRCA1-5382-InsC within human cells.

### EXAMPLE 2

This Example demonstrates generating patient-specific knock-in of BRCA1-5382-InsC in human iPSCs.

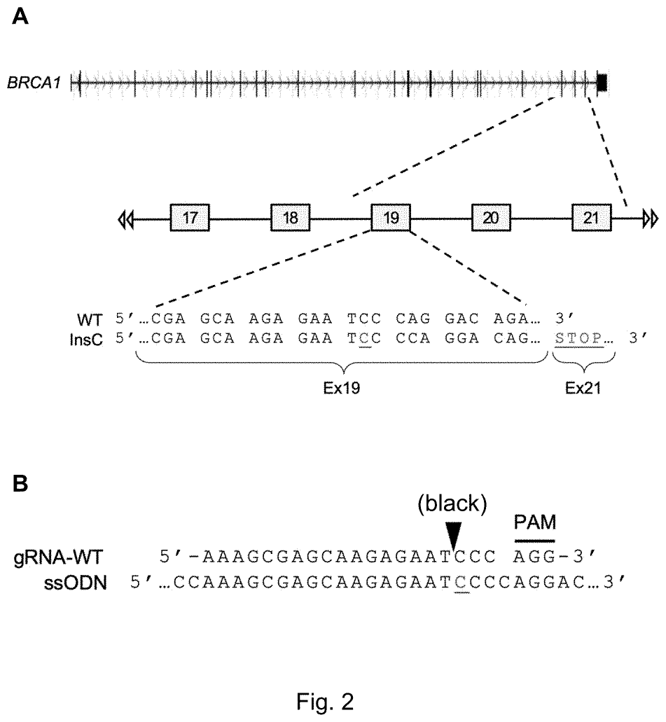

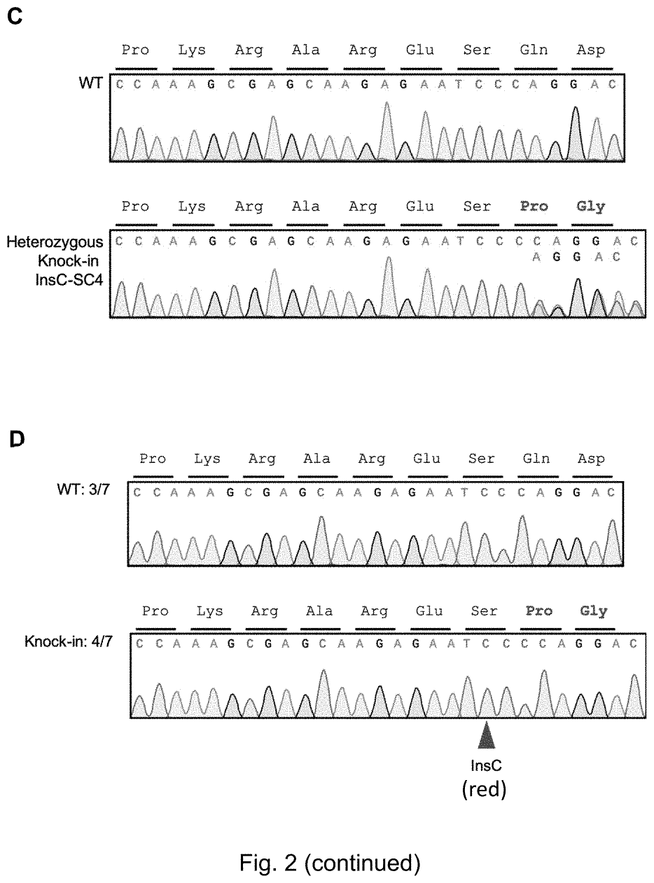

We generated a patient-specific knock-in model of BRCA1-5382-InsC, in which to perform the correction (FIG. 2, panel A). We designed a gRNA targeting the region of interest in BRCA1 (termed “gRNA-WT”), using an NGG PAM positioned 3 base pairs upstream of the target site. A 180 base pair single-stranded oligodeoxynucleotide (ssODN) containing the BRCA1-5382-InsC sequence was used as the knock-in DNA template (FIG. 2, panel B). gRNA-WT was cloned into a Cas9 vector expressing eGFP (termed “knock-in plasmid”.) Wild-type (WT) iPSCs were co-transfected with the knock-in plasmid and ssODN to generate the mutation of interest via HDR-mediated gene editing. A vector containing only eGFP (termed “control plasmid”) was co-transfected into WT iPSCs with the ssODN. Transfected cells were sorted into eGFP+ single clones, which were cultured for approximately 2 weeks. DNA was extracted, PCR-amplified, and sent for Sanger sequencing. Sequencing results revealed the presence of a WT sequence corresponding to a single clone from the control plasmid transfection. In contrast, the results corresponding to a single clone from the knock-in plasmid transfection show the successful generation of a heterozygous knock-in single clone (termed “Heterozygous Knock-in InsC-SC4”) (FIG. 2, panel C). These results indicate that the described system successfully generated a model of BRCA1-5382-InsC in iPSCs.

The genomic polymerase chain reaction (PCR) products from BRCA1 exon 19 were cloned and sequenced to validate the generation of a heterozygous knock-in. Our PCR product from Heterozygous Knock-in InsC-SC4 was transformed in TOP10 bacteria, which were then incubated in the presence of X-gal. Transformed bacteria containing recombinant vectors give rise to white clones, whereas those transformed with TOPO lacking the insert, produce blue clones. Seven white clones, each representing a single allele, were selected, purified, and sent for Sanger Sequencing. Sequencing results for individual alleles indicate that 3/7 clones contain the WT sequence and 4/7 clones contain the knock-in sequence (FIG. 2, panel D). The existence of both WT and mutated alleles in similar proportions further supports that a patient-specific heterozygous knock-in was generated.

### EXAMPLE 3

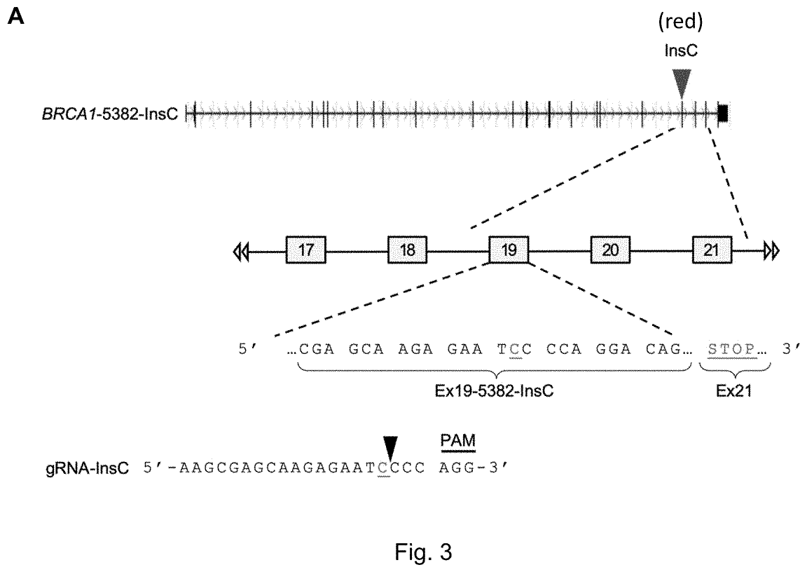

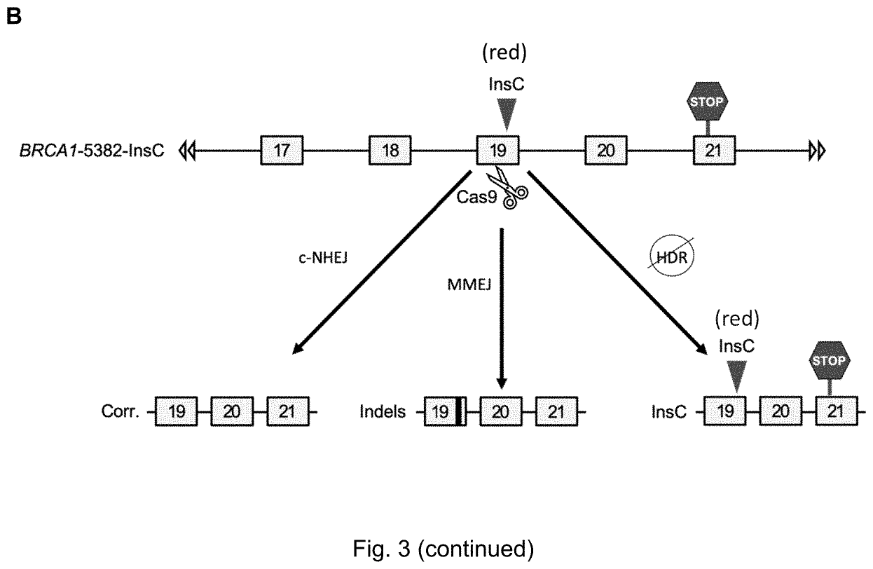

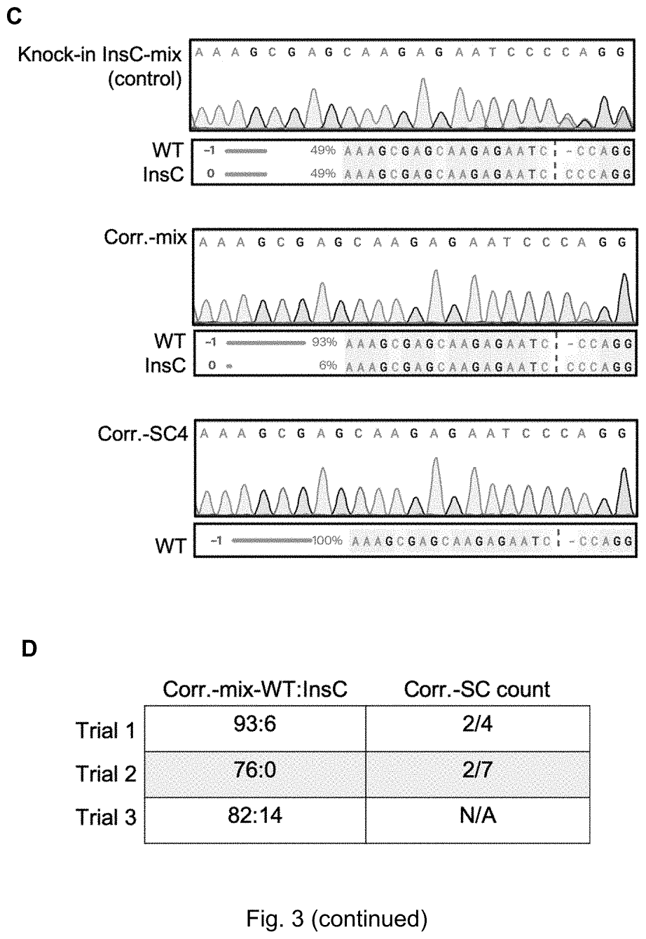

We corrected Heterozygous Knock-in InsC-SC4 in a template-free manner via the c-NHEJ pathway. gRNA-InsC was generated as described for FIG. 3, panel A. The rescue plasmid, which was also generated as described above, was transfected into Heterozygous Knock-in InsC-SC4 iPSCs. Following gRNA-InsC-mediated localization to the target site, Cas9 induces a DNA DSB between the 3rd and 4th base pair upstream of the PAM. Upon detection of the DSB, the cell initiates a repair pathway to correct the damage. The c-NHEJ pathway results in direction ligation of the broken strands and removes the mutation to rescue BRCA1. The MMEJ pathway generates indels (>2 bp) within the target region. Similarly, the HDR pathway is inhibited by the partial absence of BRCA1, which plays an essential role in HDR activation (FIG. 3, panel B).

After ˜2 weeks of culturing the transfected cells, DNA was extracted, PCR-amplified, and sent for Sanger sequencing. Sequencing results revealed the efficacy of correcting BRCA1-5382-InsC in iPSCs using our rescue plasmid. Within the control cell mixture, approximately 50% of the alleles were WT and 50% were mutated. These allelic ratios are consistent with those of Heterozygous Knock-in InsC-SC4, indicating that no editing occurred in the control sample. In contrast, the mixture of cells transfected with the rescue plasmid contained approximately 93% WT alleles and 6% mutant alleles. The increased ratio of WT:mutant alleles generated by rescue plasmid transfection, in comparison to those in the control, indicates successful, template-free correction of BRCA1-5382-InsC. Similarly, a single clone transfected with the rescue plasmid contained 100% WT alleles, further substantiating correction of the mutant allele (FIG. 3, panel C).

To further validate these results, we performed the correction in 3 independent trials(n=3). In all 3 trials, the ratio of WT:mutant alleles in the cell mixture transfected with the rescue plasmid, exceeded the ˜50:50 ratio present in Heterozygous Knock-in InsC-SC4. In trials 1 and 2, sequencing results corresponding to single clones transfected with the rescue plasmid, revealed corrected clones (FIG. 3, panel D). Together, these results indicate that BRCA1-5382-InsC was precisely corrected in iPSCs in the absence of a donor template.

### EXAMPLE 4

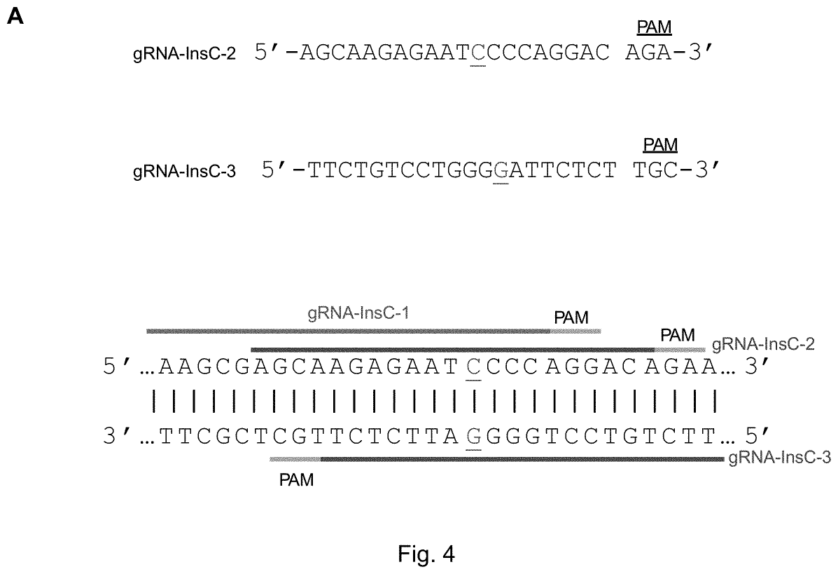

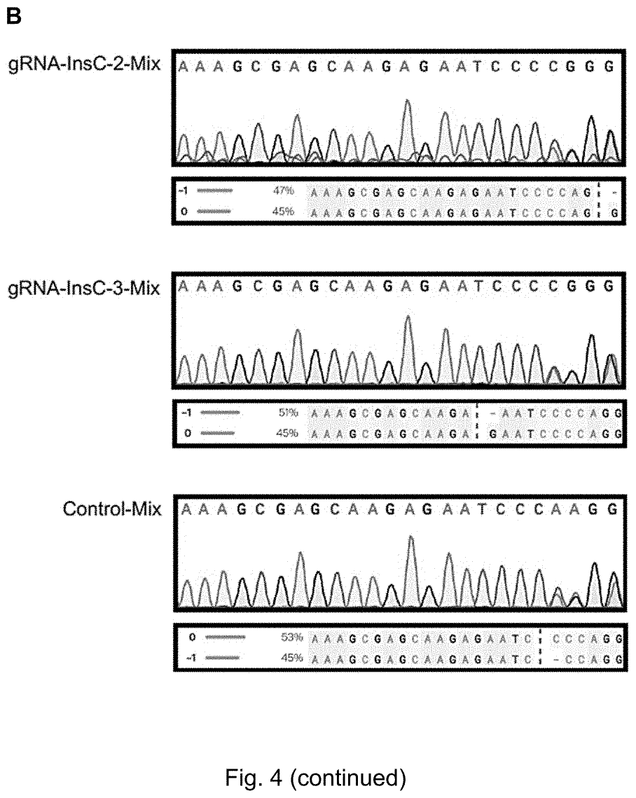

This Example relates to FIG. 4 panel A and FIG. 4, panel B, which show results from testing a gRNA pool for template-free correction of knock-in BRCA1-5382-InsC via c-NHEJ-mediated gene editing.

In FIG. 4, panel A, the 20 base pair gRNA target sequences of BRCA1-5382-InsC, labeled “gRNA-InsC-2” (upper sequence) and “gRNA-InsC-3” (lower sequence) are shown. The InsC mutation is marked in red and underlined and the PAM is labeled. The schematic shows the forward and reverse strands of the target sequence (top and bottom strand, respectively). gRNA-InsC-2 and gRNA-InsC-3 target the forward and reverse strands of DNA, respectively. gRNA-InsC-1 is included for reference. The PAM sequence for each gRNA is labeled and marked in grey, and the InsC mutation is underlined.

In FIG. 4, panel B, Sanger sequencing results (upper boxes) and corresponding ICE analyses (lower boxes) following transfection of Het-Kl-InsC-SC4 with a plasmid containing either gRNA-InsC-2 (upper sample), gRNA-InsC-3 (middle sample), or no gRNA (lower sample, labeled “Control-Mix”). The ICE analyses show the allele percentages in the cell mixture for each sample, and indicate that the corrected, WT allele is not detected in either gRNA-InsC-2-Mix or gRNA-InsC-3-Mix.

Table A provides the gRNA sequences and PAMs used for the pool of gRNAs targeting the InsC mutation.

TABLE A

| | gRNA sequence | PAM |
|---|---|---|
| gRNA-InsC-1 | 5’-AAGCGAGCAAGAGAATCCCC-3’ (SEQ ID NO: 11) | AGG |
| gRNA-InsC-2 | 5’-AGCAAGAGAATCCCCAGGAC-3’ (SEQ ID NO: 4) | AGA |
| gRNA-InsC-3 | 5’-TTCTGTCCTGGGGATTCTCT-3’ (SEQ ID NO: 5) | TGC |

All gRNAs use the following scaffold (SEQ ID NO: 6): 5′-gttttagagctagaaatagcaagttaaaataaggctagtccgttatcaacttgaaaaagtggcaccgagtcggtgc-3′ (where T is replaced by U (SEQ ID NO: 7)).

### Supplementary Tables

TABLE S1

| | | |
|---|---|---|
| Primers for | BRCA1 238F | Acctcctgcattcaaaagattctc |
| | | (SEQ ID NO: 8) |
| genotyping | BRCA1 983R | Agcaagagagagagagagaaagaca |
| | | (SEQ ID NO: 9) |
| gRNA target | BRCA1 | AAAGCGAGCAAGAGAATCCC |
| sequences | | (SEQ ID NO: 10) |
| (InsC in bold) | Ex19-5382-InsC | AAGCGAGCAAGAGAATCCCC |
| | | (SEQ ID NO: 11) |

| | |
|---|---|
| ssODN used as | tttggtttctttcagCATGATTTTGAAGTCAGAGGAGATGTGGTCAATGG |
| template to | AAGAAACCACCAAGGTCCAAAGCGAGCAAGAGAATCCCCAGGACAGAAAG |
| knock-in InsC | gtaaagctccctccctcaagttgacaaaaatctcaccccaccactctgta |
| | ttccactcccctttgcagagatgggccgct |
| | (SEQ ID NO: 12) |
| Ex19-5382-insC | CATGATTTTGAAGTCAGAGGAGATGTGGTCAATGGAAGAAACCACCAAGG |
| used to model | TCCAAAGCGAGCAAGAGAATCCCCAGGACAGAAAG |
| InsC mutation in | (SEQ ID NO: 13) |
| reporter system | |
| InsC in bold | |
| Reporter system | GTGATGCGGTTTTGGCAGTACATCAATGGGCGTGGATAGCGGTTTGACTC |
| used to test | ACGGGGATTTCCAAGTCTCCACCCCATTGACGTCAATGGGAGTTTGTTTT |
| correction | GGCACCAAAATCAACGGGACTTTCCAAAATGTCGTAACAACTCCGCCCCA |
| efficiency of | TTGACGCAAATGGGCGGTAGGCGTGTACGGTGGGAGGTCTATATAAGCAG |
| rescue plasmid | AGCTGGTTTAGTGAACCGTCAGATCCGCTAGCCACCAATG [symbol] |
| Uppercase italics: | [symbol] [symbol] [symbol] [symbol] |
| promoter | [symbol] [symbol] ACCGGTCgtgageaagggcgaggag |
| Uppercase bold | Gtcatcaaagagttcatgcgcttcaaggtgcgcatggagggctccatgaa |
| italcis: Ex19-5382- | Cggccacgagttcgagatcgagggcgagggcgagggccgcccctacgagg |
| InsC | Gcacccagaccgccaagctgaaggtgaccaagggcggccccctgcccttc |
| Lowercase bold: | Gcctgggacatcctgtccccccagttcatgtacggctccaaggcgtacgt |
| tdTom | Gaagcaccccgccgacatccccgattacaagaagctgtccttccccgagg |
| | Gcttcaagtgggagcgcgtgatgaacttcgaggacggcggtctggtgacc |
| | Gtgacccaggactcctccctgcaggacggcacgctgatctacaaggtgaa |
| | gatgcgcggcaccaacttcccccccgacggcc |
| | ccgtaatgcagaagaagaccatgggctgggaggcctccaccgagcgcctg |
| | tacccccgcgacggcgtgctgaagggcgagatccaccaggccctgaagctg |
| | aaggacggcggccactacctggtggagttcaagaccatctacatggccaag |
| | aagcccgtgcaactgcccggctactactacgtggacaccaagctggacat |
| | cacctcccacaacgaggactacaccatcgtggaacagtacgagcgctccg |
| | agggccgccaccacctgttcctggggcatggcaccggcagcaccggcagc |
| | ggcagctccggcaccgcctcctccgaggacaacaacatggccgtcatcaaa |
| | gagttcatgcgcttcaaggtgcgcatggagggctccatgaacggccacga |
| | gttcgagatcgagggcgagggcgagggccgcccctacgagggcacccaga |
| | ccgccaagctgaaggtgaccaagggcggccccctgcccttcgcctgggac |
| | atcctgtccccccagttcatgtacggctccaaggcgtacgtgaagcaccc |
| | cgccgacatccccgattacaagaagctgtccttccccgagggcttcaagt |
| | gggagcgcgtgatgaacttcgaggacggcggtctggtgaccgtgacccag |
| | gactcctccctgcaggacggcacgctgatctacaaggtgaagatgcgcgg |
| | caccaacttcccccccgacggccccgtaatgcagaagaagaccatgggct |
| | gggaggcctccaccgagcgcctgtacccccgcgacggcgtgctgaagggc |
| | gagatccaccaggccctgaagctgaaggacggcggccactacctggtgga |
| | gttcaagaccatctacatggccaagaagcccgtgcaactgcccggctact |
| | actacgtggacaccaagctggacatcacctcccacaacgaggactacacc |
| | atcgtggaacagtacgagcgctccgagggccgccaccacctgttcctgta |
| | cggcatggacgagctgtacaaagcggccgca |
| | (SEQ ID NO: 14) |

Notes:
(uppercase = exonic, lowercase = intronic)

### REFERENCES—THIS REFERENCE LISTING IS NOT AN INDICATION THAT ANY REFERENCE IS MATERIAL TO PATENTABILITY

1. Jinek, M. et al. A programmable dual-RNA-guided DNA endonuclease in adaptive bacterial immunity. Science 337, 816-821, doi:10.1126/science.1225829 (2012).
2. Huszno, J., Kolosza, Z. & Grzybowska, E. BRCA1 mutation in breast cancer patients: Analysis of prognostic factors and survival. Oncol Lett 17, 1986-1995, doi:10.3892/01.2018.9770 (2019).
3. Chen, C. C., Feng, W., Lim, P. X., Kass, E. M. & Jasin, M. Homology-Directed Repair and the Role of BRCA1, BRCA2, and Related Proteins in Genome Integrity and Cancer. Annu Rev Cancer Biol 2, 313-336, doi:10.1146/annurev-cancerbio-030617-050502 (2018).
4. Nelson, H. D. et al. Risk assessment, genetic counseling, and genetic testing for BRCArelated cancer in women: a systematic review to update the U.S. Preventive Services Task Force recommendation. Ann Intern Med 160, 255-266, doi:10.7326/M13-1684 (2014).
5. Rebbeck, T. R. et al. Breast cancer risk after bilateral prophylactic oophorectomy in BRCA1 mutation carriers. J Natl Cancer Inst 91, 1475-1479, doi:10.1093/jnci/91.17.1475 (1999).

What is claimed is:

1. A method for modifying DNA comprising a BRCA1-5382-InsC mutation, the method comprising introducing into cells comprising the BRCA1-5382-InsC a Cas enzyme and a guide RNA, said method being DNA template free, and said guide RNA being functional with an AGG protospacer adjacent motif (PAM) that is proximal to the BRCA1-5382-InsC mutation, and wherein the BRCA1-5382-InsC mutation is eliminated after introduction of the Cas enzyme and the guide RNA.

2. The method of claim 1, wherein the Cas enzyme comprises a Cas9 enzyme.

3. The method of claim 2, wherein the guide RNA comprises the sequence
(SEQ ID NO: 1)
AAGCGAGCAAGAGAAUCCCC

4. The method of claim 3, wherein the cells comprising the BRCA1-5382-InsC are breast cancer cells, ovarian cancer cells, prostate cancer cells, or melanoma cells.

5. The method of claim 4, wherein the cells are breast cancer cells.

6. The method of 5, wherein the cells are present in an individual.

7. The method of claim 6, wherein the individual has been diagnosed with breast cancer.

8. The method of claim 7, wherein the Cas9 enzyme and the guide RNA are encoded by a single expression vector that is introduced into the cells.

9. The method of claim 8, wherein the BRCA1-5382-InsC mutation is eliminated by Canonical Non-homologous end joining (c-NHEJ).

10. The method of claim 9, wherein elimination of the BRCA1-5382-InsC mutation reverses a loss of heterozygosity.

11. An expression vector encoding a guide RNA comprising the sequence AAGCGAGCAAGAGAAUCCCC (SEQ ID NO: 1), wherein the expression vector further encodes a Cas nuclease, said Cas nuclease optionally being Cas9.

12. Modified stem cells comprising an introduced BRCA1-5382-InsC mutation.

13. The stem cells of claim 12, wherein the stem cells comprise human induced pluripotent stem cells (iPSCs).

14. The stem cells of claim 13, wherein the introduced the BRCA1-5382-InsC mutation is a heterozygous mutation.

15. The stem cells of claim 15, wherein the stem cells are present in an in vitro cell culture.
